(19) United States
(12) Patent Application Publication
(10) Pub. No.: US 2024/0023850 A1
(43) Pub. Date: Jan. 25, 2024

(54) CAPILLARY BLOOD SAMPLING

(71) Applicants: Niclas Roxhed, Bromma (SE); Mikolaj Dobielewski, Stockholm (SE); Göran Stemme, Lidingö (SE)

(72) Inventors: Niclas Roxhed, Bromma (SE); Mikolaj Dobielewski, Stockholm (SE); Göran Stemme, Lidingö (SE)

(21) Appl. No.: 18/249,196

(22) PCT Filed: Oct. 14, 2021
(86) PCT No.: PCT/EP2021/078405

§ 371 Date: Apr. 14, 2023

(30) Foreign Application Priority Data
Oct. 15, 2020 (SE) 2030310-3

(51) Int. Cl. A61B 5/15; A61B 5/151

(52) U.S. Cl. CPC A61B 5/150022; A61B 5/150175; A61B 5/15045; A61B 5/150458; A61B 5/150465; A61B 5/150984; A61B 5/15111; A61B 5/15117; A61B 5/15144

(57) ABSTRACT

A microneedle (100) for extracting a blood sample from the skin (10) of a mammal subject is disclosed. The microneedle comprises at least one substantially flat blade (110) comprising at least one cutting edge (120a, 120b) configured to incise the skin of the mammal subject, and at least one microchannel (130) comprising an opening (132) and a passage (134). The at least one microchannel is arranged adjacent to the at least one blade and configured to be inserted into the skin of the mammal subject together with the at least one blade. The passage is configured to transport the blood sample away from the opening via capillary action. A device (200) for extracting a blood sample from a mammal subject comprising a microneedle is also disclosed.

### TECHNICAL FIELD

The present disclosure generally relates to the field of sampling blood, and in particular to techniques for extracting a blood sample from the skin of a mammal subject.

### BACKGROUND

To access information on the health of a patient beyond assessment of the symptoms, analyses of bodily samples need to be performed. Assessment of the concentration or presence of specific analytes in the body is an important aspect of medical practice for patient monitoring and disease diagnostics. Such an assessment allows for doctors and nurses to acquire information, either qualitative or quantitative, on the status of a patient and take medical decision based on thereon.

Depending on the disease or the condition to be assessed, different bodily matrices may be used, including tissue (for example via biopsy) and different fluids (both liquids and gases). Generally, it is common to use blood as a measurement matrix for a large variety of analytes, such as glucose, cholesterol, medical drugs, and the like.

Today different methods for blood extraction are used. Two common practices are finger pricking or intravenously extracting blood. Finger pricking generally includes a device for pricking the finger, for example with a sharp lancet. The blood surfacing is then aspirated using a separate blood-aspirating component so that it can be analyzed. However, the process is inefficient and creates a lot of dead volumes.

Intravenous blood extraction includes reaching a vein with a needle which may cause stress to a patient and unnecessary harm. Blood extraction in general is associated with invasive and painful methods, whether done via finger pricking or intravenously.

Thus, there is a need for improved sampling techniques in general, and for an improved device for extracting a blood sample in particular.

### SUMMARY

It would be advantageous to achieve a technique overcoming or alleviating at least some of the above-mentioned drawbacks. In particular it would be desirable to enable an improved device for extracting a blood sample from a mammal subject.

Blood samples are widely used in medical diagnostics, making blood drawing one of the most common invasive procedures in health care. The required volume of the sample is largely dictated by the sensitivity of the analysis performed on the sample. The advancements in the field of a liquid chromatography-tandem mass spectrometry (LC-MS/MS) have enabled quantitative analysis of single microliters of biological samples. This allows reducing the blood sample volume by an order of magnitude with respect to the well-established finger-pricking, which leads to further reduction in the invasiveness and pain of minimally invasive blood sampling. Solid microneedles have been successfully used to sample capillary blood, but they lack the possibility to directly aspirate blood, and need to be accompanied by separate blood-aspirating components. This makes the complete sampling device procedure less efficient, due to larger dead volumes, which result in larger minimal blood volume which can be reliably sampled. Additionally, no fully integrated device for dry blood spot (DBS) capillary blood sampling has been presented to date. DBS samples can be safely transported by post to a centralized laboratory without the risk of exposing postal workers to potentially dangerous liquid samples. DBS samples can also be stored without temperature control and analyzed even years after collection. A device painlessly collecting the minimal blood volume in a simple, one-step manner and seamlessly producing a DBS sample has the potential to improve the comfort of patients and their access to high quality, patient-centric diagnostics.

To better address one or more of these concerns, a microneedle and a device having the features defined in the independent claims are provided. Preferable embodiments are defined in the dependent claims.

Hence, according to a first aspect, a microneedle for extracting a blood sample from the skin of a mammal subject is provided, comprising at least one substantially flat blade having at least one cutting edge, wherein the at least one cutting edge is configured to incise the skin of the mammal subject. The microneedle further comprises at least one microchannel having an opening and a passage, wherein the at least one microchannel is arranged adjacent to the at least one blade and configured to be inserted into the skin of the mammal subject together with the at least one blade. The passage is configured to transport the blood sample away from the opening via capillary action.

According to a second aspect, a device for extracting a blood sample from a mammal subject is provided. The device comprises a microneedle according to the first aspect of the invention. The device further comprises a body configured to support the microneedle, a channel arranged in the body and fluidically connected to the passage of the microneedle, and a retaining material arranged in the body. The retaining material is fluidically connected to the channel and configured to absorb and store the blood sample transported by the passage of the microneedle to the channel.

The above microneedle and device may further be used in a method in which the microneedle is inserted into the skin of the mammal subject and the blood sample is extracted to a retaining material, in which the blood sample is absorbed and stored. The extraction may be facilitated by the microchannel acting like a capillary channel and aspirating the blood sample from the skin of the mammal subject.

The blood sample, stored in the retaining material, may then be subject to biological or chemical analysis, e.g. performed by standard laboratory tools such as mass spectrometry. The chemical analysis may for example be performed to detect drugs of abuse, such as phosphatidylethanol, amphetamine, MFM, MDMA, tetrahydrocannabinol (THC), cocaine and opiates. The analysis may be performed for quantification purposes or for merely indicating presence of these substances.

The microneedle may be understood as a micro-scaled needle which due to its small dimensions can be inserted into the skin relatively painlessly and without causing any significant tissue damage, especially as compared to conventional hypodermic needles. The device may comprise a plurality of microneedles, for example arranged in two-dimensional arrays. The microneedle may be formed from a variety of materials such as silicon or glass, metals such as stainless steel, or polymers such as for example a hydrophilic polymer. Preferably, the microneedle has a length that allows its tip to be arranged in the dermis of the skin of the mammal subject. Hence, it is understood that the dimensions of the microneedle may vary depending on the sampling location and the actual thickness of the skin at the sampling location. Various examples and dimensions of the microneedle will be discussed later in connection with the description of different embodiments.

The microneedle may be configured to penetrate the epidermis and provide access to the blood in the dermal region. Due to its relatively small size, the use of the microneedle may be relatively painless both during insertion and after removal, potentially improving patient acceptance and compliance. Moreover, the use of a microneedle has shown to reduce the risk for long term damage to the skin, which makes it an attractive technique for repeated sampling compared to collecting venous blood or finger pricking.

The at least one blade may be fixed in relation to the at least one microchannel. Thus, the at least one microchannel may automatically be inserted into the skin of the mammal subject together with the at least one blade. The present embodiment may be advantageous in that it is easy to use in order to extract a blood sample. By penetrating the skin and then slightly removing the microneedle, blood from the skin will start to flow and be aspirated by the at least one microchannel.

The at least one blade may be independently movable in relation to the at least one microchannel. The present embodiment may be preferred in different situations. The blade may be used in order to incise the skin and ensure that a blood flow may start. In an advantageous embodiment the at least one blade may then be configured to be removed from the skin of the mammal subject independently from the at least one microchannel while the at least one microchannel is aspiring blood.

The microchannel may further comprise an exit arranged at an end portion of the passage opposing the opening. The exit may comprise a cut extending along the axial direction of the passage. This may ensure that a fluid moving through the passage can more easily exit the exit of the microchannel. The cut may create at least one contact point allowing the blood sample in the microchannel to come into contact with a material outside of the microchannel. This may be advantageous in that the fluid may come into contact with something outside of the passage so that it can flow more easily and exit the passage in a dependable manner.

A diameter of the at least one microchannel may be substantially smaller than a width of the at least one blade. The blade may be large enough to cause enough damage so that bleeding starts, but it is preferably sharp and slender in order to not cause harm to the patient. The microchannel needs to be small enough in order to aspirate blood via the capillary force. In some advantageous embodiments, the at least one cutting edge may comprise an apex with a tip radius smaller than 20 μm, preferably smaller than 5 μm, more preferably smaller than 1 μm. The at least one microchannel may have an inner diameter larger than 30 μm and smaller than 500 μm. The at least one blade may have a thickness smaller than 1 mm and larger than 10 μm. The at least one blade may have a width that is larger than 500 μm, preferably larger than 1 mm. The different dimensions may vary depending on the embodiment.

The at least one blade may be formed by two sub blades, each having a cutting edge, and wherein the sub blades are joined into a tip. The blade may thusly be formed out of at least two sub blades to form an arrow-like microneedle with a tip and a cutting edge on each side of the tip. The opening of the at least one microchannel may be aligned with the tip. The microneedle may be configured so that the tip may be the first to incise the skin of the mammal subject. When the blade is slightly retracted after the incision the tip may still be in contact with the blood arising from the skin of the mammal subject. It may therefore be advantageous if the microchannel is aligned with the tip allowing it to easily aspirate the blood sample. The at least one microchannel may be arranged in-between the two sub blades so that each sub blade extends at least partially along a length direction of the microchannel and so that the opening is aligned with the tip.

The at least one microchannel is configured to aspirate the blood via capillary force. In some embodiments it may be advantageous to have a hydrophilic coating or hydrophilic fibers. Therefore, in certain advantageous embodiments the passage may comprise a hydrophilic coating. In another embodiment at least one wall of the channel of the device may comprise a hydrophilic coating. It is further envisioned that the device may comprise hydrophilic fibers extending from the channel into the passage.

The microneedle may also have more than one blade. The microneedle may comprise a plurality of substantially flat blades each comprising at least one cutting edge configured to incise the skin of the mammal subject. This may be advantageous in that more blades may cause more damage and thusly a heavier blood flow which in certain embodiments may be necessary. However, the substantially flat blades may be configured to not cause substantial harm to the patient. This may be enabled by the flatness and sharpness of the blades. The microneedle may further comprise a plurality of microchannels arranged adjacent at least one blade and configured to transport the blood sample away from the skin of the mammal subject. Depending on the embodiment and usage of the microneedle different amounts of blood may be needed for a sample. A plurality of microchannels may for example increase the speed of the blood sampling. It may also facilitate taking multiple tests simultaneously. Each microchannel may also fluidically connected to a retaining material so that more than one blood sample is taken from one single wound and with only one incision.

The at least one microchannel may be a closed microchannel. The at least one microchannel may also be an open microchannel. In case the microchannel is open it may for example be a trench etched into the at least one blade. The at least one microchannel may be a lumen. The passage of the at least one microchannel may have a hollow interior, also referred to as a lumen, or a trench being arranged in an outer surface of the microchannel and extending along the length direction of the microchannel. The microneedle may also comprise a capillary means in connection to the at least one microchannel.

The device may in certain embodiments have an ejecting mechanism that is configured to move the microneedle towards and into the skin of the mammal subject. The ejecting mechanism may be configured to be triggered in response to the body of the device being pushed against the skin of the mammal subject. The ejecting mechanism may comprise a preloaded spring configured to move the microneedle when the body is pushed against the skin of the mammal subject. An ejecting mechanism may be advantageous in that a precise ejecting distance from the body of the device can be set. Depending on the use for the device blood from different depths in the skin may be needed for sampling. For example, blood from the dermis region in the skin may be wanted for sampling, an incision with a depth of at least 1 mm may then be needed. The ejecting mechanism may then be configured to move the microneedle a first distance d1 into the skin.

The device may also have a retracting mechanism configured to move the at least one blade at least partially away from the skin after the skin has been penetrated. The retracting mechanism may be configured to move the at least one blade a second distance d2 opposite the first direction d1. The first distance d1 may be equal to or larger than the second distance d2. The retracting mechanism may comprise a spring that is loaded by the movement of the ejecting mechanism and in response moves at least part of the microneedle at least partially away from the skin of the mammal subject. It may be advantageous to have a retracting mechanism that at least moves part of the microneedle away from the skin of the mammal subject after the incision. The at least one blade is used in order to incise the skin and to start the bleeding, but in case the blade is kept stationary it may disable the aspiration of blood. In case the blade is retracted slightly it allows the blood to flow from the capillaries of the mammal subject and be aspirated by the at least one microchannel.

Other objects, features and advantages of the enclosed embodiments will be apparent from the following detailed description, from the attached dependent claims as well as from the drawings. Those skilled in the art realize that different features of the present invention, even if recited in different claims, can be combined in embodiments other than those described above and in the following.

### BRIEF DESCRIPTION OF THE DRAWINGS

Exemplifying embodiments will now be described in more detail with reference to the following appended drawings, on which:

FIG. 1 is a schematic illustration of a microneedle according to an embodiment;

FIG. 2 is a schematic illustration of a microneedle according to an embodiment;

FIG. 3 is a schematic illustration of a microneedle according to an embodiment;

FIG. 4 is a schematic illustration of an exit portion of a microchannel;

FIG. 5 is a schematic illustration of a fluid exiting a microchannel according to an embodiment;

FIG. 6a-6d illustrates how a blood sample is extracted from the skin of a mammal subject using a microneedle according to an embodiment;

FIG. 7a-7d illustrates how a blood sample is extracted from the skin of a mammal subject using a microneedle according to an embodiment;

FIG. 8 is a schematic illustration of a device according to an embodiment;

FIG. 9 is a schematic illustration of a device according to an embodiment; and

FIG. 10a-10f illustrates how a blood sample is extracted from the skin of a mammal subject using a device according to an embodiment.

As illustrated in the figures, the sizes of the elements and regions may be exaggerated for illustrative purposes and, thus, are provided to illustrate the general structure of the embodiments. Like reference numerals refer to like elements throughout.

### DETAILED DESCRIPTION

Exemplifying embodiment will now be described more fully hereinafter with reference to the accompanying drawings, in which currently preferred embodiments are shown. The invention may, however, be embodied in many different forms and should not be construed as limited to the embodiments set forth herein; rather, these embodiments are provided for thoroughness and completeness, and fully convey the scope of the invention to the skilled person.

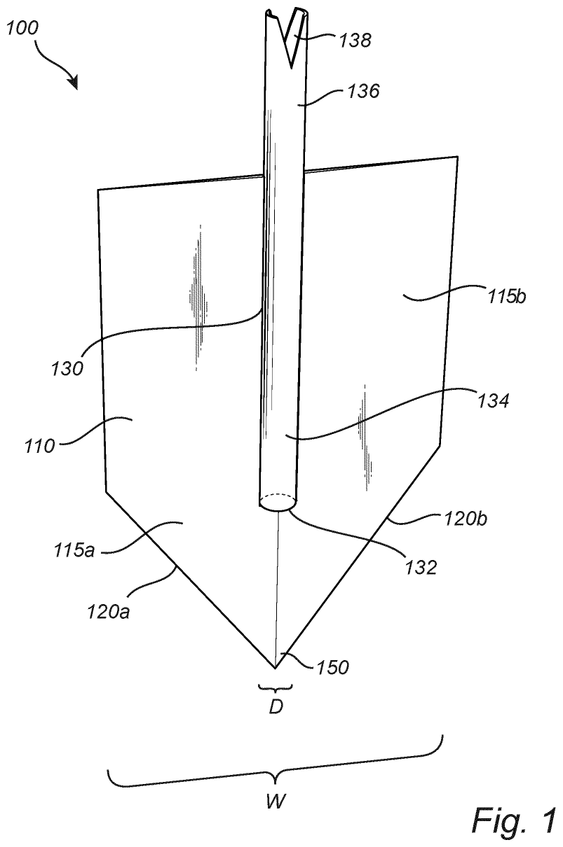

FIG. 1 is a schematic illustration of a microneedle 100 according to an embodiment of the invention. The microneedle 100 is configured for extracting a blood sample from the skin of a mammal subject. The microneedle 100 comprises a flat blade 110 with a cutting edge 120a, 120b which is configured to incise the skin of the mammal subject. The microneedle 100 also comprises a microchannel 130 with an opening 132 and a passage 134. The microchannel 130 is configured to be inserted into the skin of the mammal subject together with the blade 110. The passage 134 is configured to transport the blood sample away from the opening 132 via capillary action.

The microneedle 100 may be inserted into the skin of a mammal subject so that blood is drawn. The blood may then be aspirated through the microchannel 130 in order to extract the blood sample from the mammal subject.

In FIG. 1 the microchannel 130 also comprises an exit 136 arranged at an end portion of the passage 134 opposing the opening 132. The exit 136 comprises a cut 138 extending along the axial direction of the passage 134. The cut 138 may be made such that blood, or any other fluid, reaching the exit 136 after traveling through the passage 134 can come into contact with a material outside of the microchannel 130. The microchannel may for example be in physical contact with an enclosing channel fluidically connected to a retaining material. By allowing the blood flowing through the passage 134 to come into contact with the enclosing channel the surface tension at the exit 136 may be counteracted and the blood or fluid may flow out from the microchannel 130. The cut 138 may have any suitable shape, for example a V- or U-shape.

The flow of blood may be facilitated or driven by capillary action of the passage 134. The passage 134 or any other part of the microneedle 100 may also comprise a hydrophilic coating or have hydrophilic fibers in order to enable a steady flow.

The blade 110 of the microneedle 100 of FIG. 1 comprises two sub blades 115a, 115b. The sub blades 115a, 115b each has a cutting edge 120a, 120b and are joined together into a tip 150. The tip 150 may be configured to be the first part of the microneedle 100 to incise the skin. The microchannel 130 may be aligned with the tip 150 so that when blood starts to flow the blood is directed towards the microchannel 130.

The microneedle 100 may have different dimensions depending on the use for the microneedle 100. It is however preferable that the blade 110 is flat and small, however it needs to be wide enough to achieve enough damage to start the flow of blood. The microchannel 130 preferably has dimensions allowing it to transport the blood via capillary action. The cutting edge 120a, 120b of the blade 110 may have an apex with a tip radius smaller than 20 μm, preferably smaller than 5 μm, more preferably smaller than 1 μm. The microchannel 130 may have an inner diameter D larger than 30 μm and smaller than 500 μm. The blade 110 may have a thickness smaller than 1 mm and larger than 10 μm. The width W of the blade 110 may be larger than 500 μm, preferably larger than 1 mm. The cutting edge 120a, 120b of the blade may be configured to create an incision on the skin surface of a length less than 5000 μm, typically less than 1500 μm and larger than 100 μm.

The microchannel 130 may be a closed microchannel 130. The at least one microchannel 130 may also be an open microchannel 130. In case the microchannel 130 is open it may for example be a trench etched into the at least one blade 110. The microchannel 130 may be symmetrical and have a circular cross-section. A cross-section of the microchannel 130 may also be elliptical or any other suitable shape.

The blood sample that is to be extracted using the microneedle 100 may have a volume less than 100 μL, typically less than 10 μL. For example, the volume of the blood sample may be 1 μL. The blood sample may be transported, aspirated, by the microchannel 130 to a retaining material for storing the blood. It is preferable to only extract a small volume of blood since the cut needed is smaller and patient will not experience that much pain.

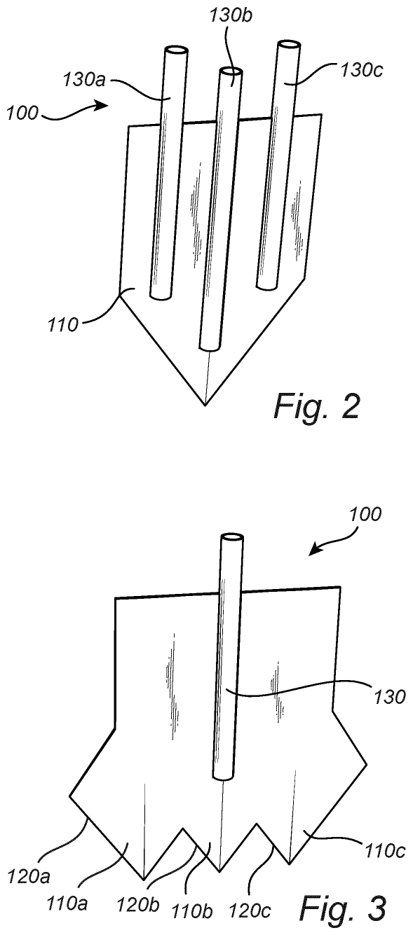

FIG. 2 is a schematic illustration of a microneedle 100 according to an embodiment of the invention. The microneedle 100 may have any of the features presented in relation to FIG. 1. The microneedle 100 differs from the disclose of FIG. 1 in that it comprises a plurality of microchannels 130a, 130b, 130c. The microchannels 130a, 130b, 130c are arranged adjacent the blade 110 and configured to transport the blood sample away from the skin of the mammal subject.

In FIG. 2 three microchannels 130a, 130b, 130c are present. There may be more or fewer than three. More than one microchannel 130a, 130b, 130c may be used in order to obtain more than one blood sample from a single microneedle 100 and a single incision. By allowing each microchannel 130a, 130b, 130c to aspirate blood into individual retaining materials more than one blood sample can be obtained.

FIG. 3 is a schematic illustration of a microneedle 100 according to an embodiment of the invention. The microneedle 100 may have any of the features presented in relation to FIG. 1 or 2. The microneedle 100 differs from the disclosure of FIGS. 1 and 2 in that it comprises a plurality of substantially flat blades 110a, 110b, 110c. Each blade comprises at least one cutting edge 120a, 120b, 120c configured to incise the skin of the mammal subject. The blades 110a, 110b, 110c may be used in certain embodiments to cause enough damage in order for the microneedle 100 to extract a blood sample of sufficient volume. The illustration in FIG. 3 is merely an example of how the microneedle 100 may look, the plurality of blade 110a, 110b, 110c may be more or fewer than three and shaped in many different ways. The microneedle 100 may also be configured to create one incision or more than one incision. The embodiment of FIG. 3 may further comprise more than one microchannel 130 for aspirating the blood sample or samples.

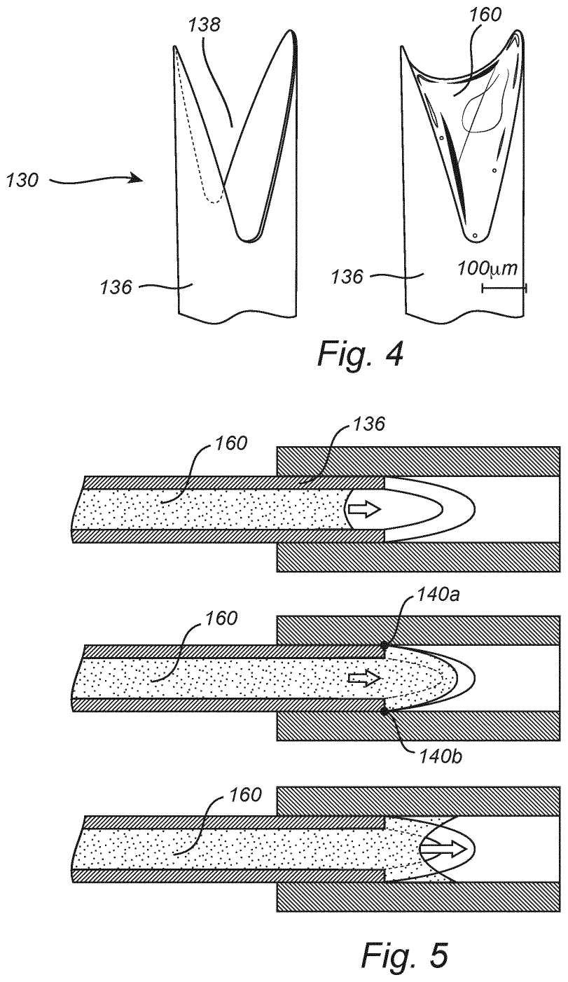

FIG. 4 is a schematic illustration of an exit 136 of a microchannel 130. The microchannel 130 may for example be used in a microneedle in accordance with the present invention. However, the microchannel 130 comprising the exit 136 may also be used in other product or environments.

The microchannel 130 comprises an exit 136 arranged at an end portion of a passage of the microchannel opposing a possible opening of the microchannel 130. The exit 136 comprises a cut 138 extending along the axis direction of the passage and microchannel.

The cut 138 may create at least one contact point allowing a fluid in the microchannel 130 to come into contact with a material outside of the microchannel 130. As an example, in case the microchannel 130 is used in a microneedle according to any one of the previous figures a blood sample may be flowing through the microchannel 130 due to capillary action. Once the blood sample reaches the exit 136 the blood can then due to the cut 138 come into contact with a material outside of the microchannel 130. This can enable an easy flow of the blood through the microchannel 130.

The cut 138 may be a V-shaped cut. It may also have any other suitable three-dimensional geometry that allows the fluid transported through the microchannel 130 to come into contact with a material outside of the microchannel 130. For example, the cut 138 may be U-shaped or C-shaped.

FIG. 5 is a schematic illustration of a fluid 160 exiting a microchannel 130 according to an embodiment. The fluid 160 may be any type of fluid transported through the microchannel 130. The fluid 160 may for example be blood transported through microchannel 130 via capillary action. Depending on the dimensions of the microchannel 130 the fluid 160 may be transported using only capillary action. The microchannel 130 may also be coated with a hydrophilic coating in order to further facilitate the transportation of the fluid 160 through the microchannel 130.

In a first step in FIG. 5 a fluid 160 is aspirated into the microchannel 130. For example, this may be blood aspirated into the microchannel 130 after being used in a microneedle described with regards to the previous figures. In a second step the fluid 160 reaches an exit 136 of the microchannel 130. A cut 138 in the exit 136 has created two contact points 140a, 140b where the fluid 160 can come into contact with a material outside the microchannel 130. In this case, the fluid 160 comes into contact with a channel 220 outside the microchannel 130. The channel may be coated with a hydrophilic coating allowing it to further move the fluid 160. In the last step the fluid 160 can be seen exiting the microchannel 130 and continue to be aspirated into the channel 220.

In case the microchannel 130 would have had a straight cut at the exit 136 the surface tension of the fluid could hinder the fluid from exiting the microchannel 130. By creating contact points 140a, 140b with an outside channel or material the fluid can easily flow out of the microchannel 130 and thus fulfil its purpose.

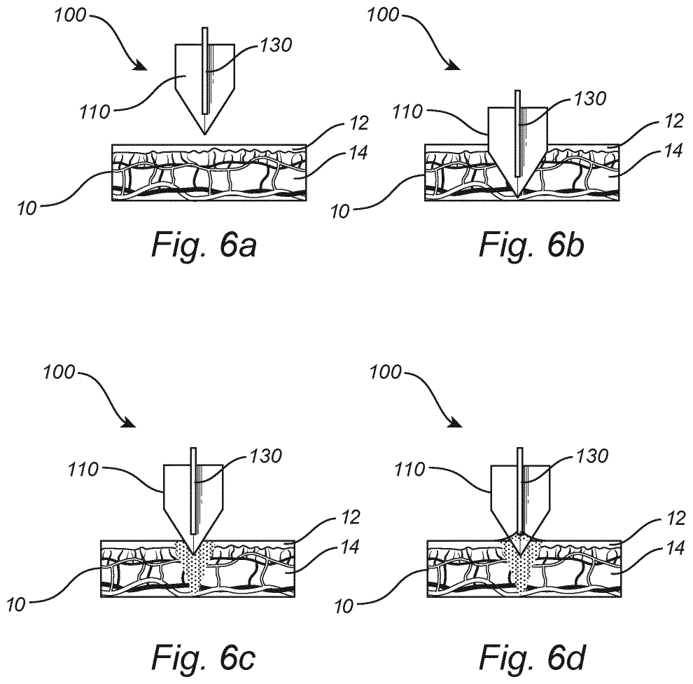

FIGS. 6a-6d illustrates how a blood sample is extracted from the skin 10 of a mammal subject using a microneedle 100.

Generally, there are two main layers of the skin 10: epidermis 12 and dermis 14. Below dermis 14, a third layer of tissue is present; the hypodermis 16 (or subcutaneous tissue). The outermost layer, the epidermis 12, serves as a waterproof barrier enclosing the body of the subject and acts as a protection against infections. The middle layer, the dermis 14, protects the body from external stress and strain, and hosts thermo- and mechanoreceptors. The subcutaneous tissue mainly consists of connective and fat tissue. Its main purposes are to attach the skin to muscles and bones, and to connect nerves and blood vessels to the skin 10. The thickness of the different layers strongly varies across different body locations and between different species, with the overall skin thickness of the human ranging from 0.05 millimeters on the eyelids to more than 1.5 millimeters on the feet soles. Considering the human forearm, the typical location used for example for blood sampling, the average skin thickness is about 1 millimeter.

In FIG. 6a the microneedle 100 is arranged in order to take a blood sample from the skin 10. In FIG. 6b the microneedle 100 is inserted into the skin 10. The blade 110 of the microneedle 100 comprises a cutting edge that is configured to incise the skin 10. The dimensions of the blade 110 and microneedle 100 in general are adjusted to decrease the harm made to the patient. The microneedle 100 is configured to be inserted a certain distance into the skin 10 in order to draw blood, preferably vascular blood in the dermis region of the skin 10.

In FIG. 6c the microneedle 100 has been slightly retracted from the wound and the skin 10. This can be done in order to allow the blood flow to start from the capillaries of the skin 10. The blood will then start to flow and rise towards the epidermis 12 and the surface of the skin 10. In FIG. 6d blood will be aspirated by the microchannel 130 and can be transported out from the skin 10 and examined in a suitable manner.

In the embodiment of the microneedle 100 in FIGS. 6a-6d the microchannel 130 and the blade 110 are fixed in relation to each other. However, the blade 110 may also be independently movable with respect to the microchannel 130.

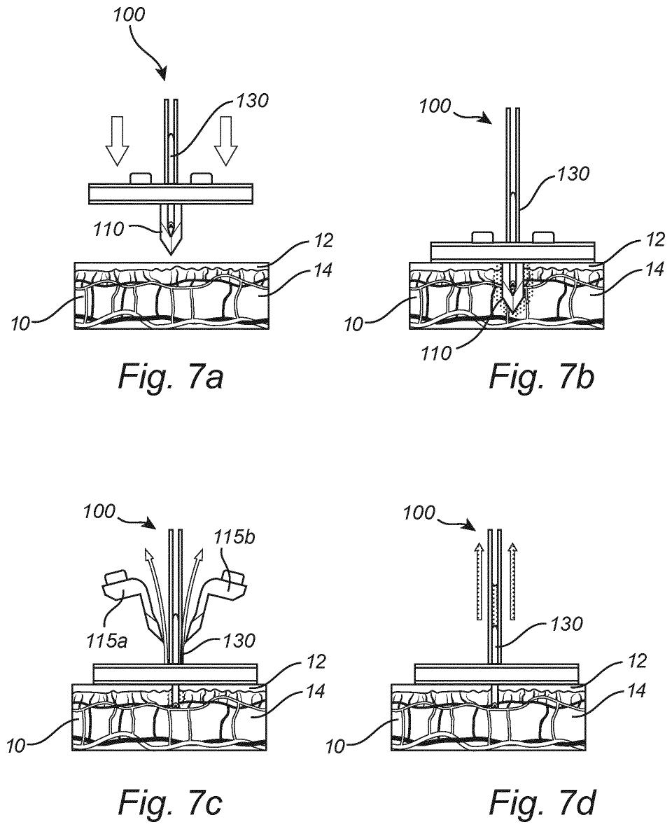

FIGS. 7a-7d illustrates how a blood sample is extracted from the skin 10 of a mammal subject using a microneedle 100. The microneedle 100 comprises a blade 110 and a microchannel 130 that are independently movable with respect to each other. In a first step illustrated in FIG. 7a the microneedle 100 is brought into the vicinity of the skin 10. In FIG. 7b the microneedle 100 is inserted into the skin 10. The microneedle 100 penetrates the skin 10 deep enough to draw blood from the capillaries of the skin 10. In FIG. 7c the blade 110 is removed from the skin 10. In this embodiment the blade 110 comprises two sub blades 115a, 115b that are removed simultaneously from the skin 10. The present embodiment is advantageous in that it allows the microchannel 130 to start aspirating blood when the blade 110 is removed. The removal of the blade 110 will ensure that the capillaries of the skin 10 are not blocked and that a blood flow can start. The blood will then reach the microchannel 130 and be aspirated up into the microchannel 130 via capillary action as depicted in FIG. 7d.

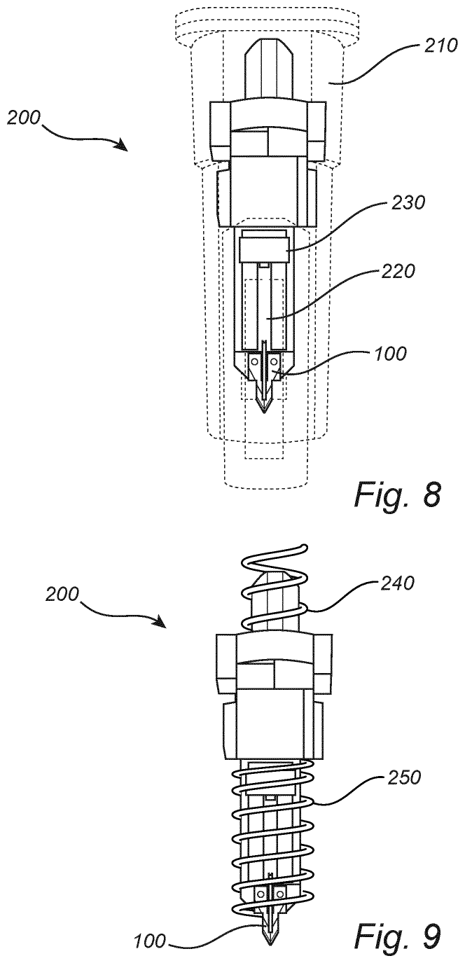

FIG. 8 is a schematic illustration of a device 200 according to an embodiment of the invention. The device 200 is configured to extract a blood sample from a mammal subject. The device 200 is configured to make an incision in the skin and aspirate blood with the integrated microneedle 100. The microneedle 100 may have any feature described in the present application text. The integrated design enables blood aspiration close to the incision site, enabling blood sampling even if only single microliters are produced. The microneedle 100 may be around 1 mm wide, 300 μm thick and may be configured to penetrate 1.5 mm into the skin. The microneedle 100 may comprise two laser-cut, stainless-steel sub blades joined together in a tip. The blade may have at least one cutting edge that cuts the skin. The microneedle 100 may also have a laser-cut stainless-steel capillary or microchannel that aspirates the blood into the device 200. The microchannel may be passivated by sonication in 10% wt. solution of citric acid to improve the capillary aspiration of blood. The microneedle 100 may also be made in other ways and by other materials.

The device 200 further comprises a body 210 configured to support the microneedle 100. The device 200 also comprises a channel 220 arranged in the body 210 and fluidically connected to the passage of the microneedle 100. Further the device 200 has a retaining material 230 arranged in the body 210. The retaining material 230 is fluidically connected to the channel 220 and configured to absorb and store the blood sample transported by the microneedle 100.

The body 210 may be made out of any suitable material, for example plastic. The body 210 may be configured to house the microneedle 100 so that it does not extend from the body 210 until it is supposed to incise the skin.

The retaining material 230 may be an analytical grade paper. For example, a dry blood spot paper such as Ahlström 222, Whatman 903, DMPK etcetera. After the blood sample has been absorbed by the retaining material the blood sample may be used for biological or chemical analysis. The chemical analysis may be performed by standard laboratory tools, such as e.g. mass spectrometry, immunoassays, suspension bead assay. It may be done to search for pharmaceutical agents, for metabolites or proteins or the like. It may also be done in order to search for drugs of abuse, for example Phosphatidylethanol, amphetamine, MDM, MDMA, Tetrahydrocannabinol (THC), cocaine, opiates.

FIG. 9 is schematic illustration of a device 200 according to an embodiment of the invention. The embodiment disclosed in FIG. 9 discloses some features that may be advantageous to incorporate into the device. The device 200 further comprises an ejecting mechanism 240 and a retracting mechanism 250. The ejecting mechanism 240 may for example be a preloaded spring 240 that is configured to launch the microneedle 100 towards the skin of the mammal subject in order to incise the skin and be able to extract a blood sample. The device 200 may further comprise a retracting mechanism 250 configured to move at least parts of the microneedle 100 away from the skin of the mammal subject after the incision is made. The retracting mechanism 250 may be configured to move at least a blade of the microneedle 100 away from the skin in order to allow blood to flow. The retracting mechanism 250 disclosed in FIG. 9 is a spring that is loaded by the ejecting mechanism 240. When the ejecting spring 240 is triggered it will move the microneedle 100 towards the skin and at the same time give the retracting spring 250 potential energy that will be released when the retracting spring moved the microneedle 100 slightly away from the skin.

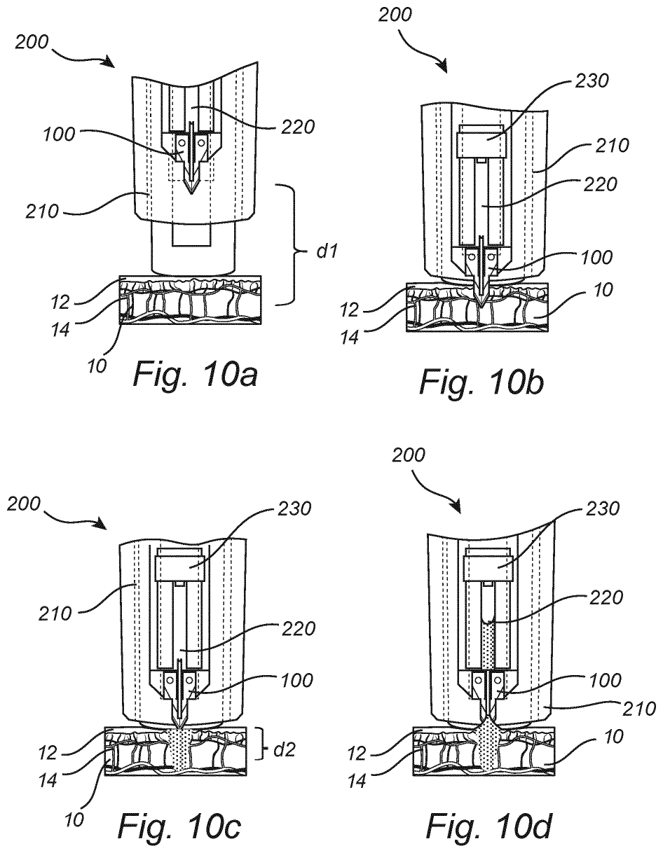

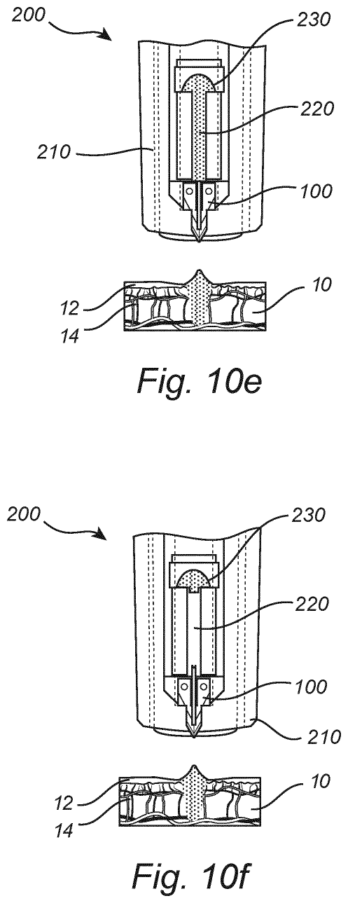

FIG. 10a-10f illustrates how a blood sample is extracted from the skin 10 of a mammal subject using a device 200 according to an embodiment. The device 200 may have any of the features presented in relation to the previous Figures.

The operation of the device 200 is presented in FIGS. 10a-10f. The device 200 is triggered by pushing it against the skin 10. The ejecting mechanism shoots the microneedle 100 towards the skin 10, making an incision. The microneedle 100 may be moved a first distance d1 towards and into the skin 10. Immediately afterwards, the retracting mechanism causes the microneedle 100 to retract from the wound a second distance d2, the microneedle 100 may be moved around 1 mm from the deepest point reached in the skin, which reduces the strain in the tissue, allowing blood to flow through the cut capillary vessels. Blood starts flowing from the cut and it is directly aspirated into the device 200. Once the channel 220 is full, the blood touches the retaining material, for example a DBS paper pad, 230 and is aspirated into it. The user removes the device 200 from the skin and the all blood present in the channel is transferred into the DBS pad.

Presented is a fully integrated device for collecting a blood sample, for example a dried blood spot sample of capillary blood in a one-step process. The blood sample may be obtained using a device 200 equipped with the microneedle 100 according to the invention. The microneedle 100, channel 220 and retaining material 230 may be configured to be in a sampling cartridge placed within the body 210 of the device 200. The microneedle 100 is configured to make an incision in the skin and aspirate blood into the channel 220 and then into the retaining material 230. The operation of the device 200 disclosed in FIGS. 10a-10f is driven by a spring mechanism triggered by a single push against the skin 10. The microneedle 100 and the blade of the microneedle is positioned with respect to the skin 10 to provide maximum blood flow from the cut tissue, minimizing the required incision size and the related pain.

The blood, which is aspirated through the microchannel of the microneedle 100, is transferred into the channel 220. The channel may have a volume of 1 μL. The capillary action may advance the blood through the microchannel and leave the microchannel due to a cut in the microchannel as disclosed within this application.

In FIG. 10a the body 210 is pressed against the skin 10 which causes the microneedle 100 to be launched into the skin 10 as seen in FIG. 10b. In FIG. 10c the retracting mechanism has moved the microneedle 100 a distance d2 away from the wound which allows a blood flow to be aspirated into the microchannel of the microneedle 100 as seen in FIG. 10d. When the channel 220 has been filled and the retaining material 230 started to absorb the blood the device 200 can be removed from the skin 10 as seen in FIGS. 10e and 10f.

Although features and elements are described above in particular combinations, each feature or element can be used alone without the other features and elements or in various combinations with or without other features and elements.

Additionally, variations to the disclosed embodiments can be understood and effected by the skilled person in practicing the claimed invention, from a study of the drawings, the disclosure, and the appended claims. In the claims, the word “comprising” does not exclude other elements, and the indefinite article “a” or “an” does not exclude a plurality. The mere fact that certain features are recited in mutually different dependent claims does not indicate that a combination of these features cannot be used to advantage.

### Experimental Results

A device according to an embodiment of the present disclosure was evaluated in a study, which is recited in the following to further exemplify embodiments and possible use of the invention. The device used corresponds to an embodiment of the device according to the present invention. The device was composed of a microneedle according to the invention and a body for supporting the microneedle. The device used in the study also had a channel arranged in the body and fluidically connected to the passage of the microneedle and a retaining material arranged in the channel.

The device further used two springs, one as ejecting mechanism and one as retracting mechanism. The ejecting mechanism was triggered by the body of the device being pushed against the skin of the mammal subject. When the ejecting mechanism was triggered it shoots the microneedle against the skin. The retracting mechanism retracted the microneedle slightly after the incision was made in order to allow the blood to flow.

The device used during the study had a housing and spring mechanism that were modified components of a VeriFine safety lancet (PromiseMed Medical Devices Inc., Canada). The device in the study used a 3D printed part, which replaced the lancing element of the VeriFine safety lancet in order to house the microneedle according to the invention. The device consisted of a cartridge for sample collection encased in a housing, body, with a spring mechanism. The cartridge was equipped with a microneedle according to the invention with a blade and a hydrophilic microchannel, and a DBS paper pad. The microneedle was 1000 μm wide, 300 μm thick and penetrated 1-1.5 mm into the skin. It consisted of two extremely sharp microblades, sub blades, (edge radius <200 nm), which cut the skin, and a steel capillary, microchannel, (OD 300 μm, ID 150 μm), which aspirated the blood into the device.

By pressing the housing against the skin, the spring mechanism was released, shooting the cartridge with the microneedle towards the skin. Immediately an incision in the skin was made, the cartridge was then automatically retracted, which relaxes the skin. This drastically improves the blood flow in the cut. The microneedle tip then rested on the skin surface while the surfacing blood was aspirated into the device through the capillary channel of the microneedle.

The study achieved reliable dried blood spot sample collection in-vitro. Aspiration of 1 μL of whole blood (C_{Hb}=140 g/L) took on average 17.8 s (SD s, n=3).

The device has also been tested in-vivo, on a pilot group of volunteers, with repeated success. The device was tested on 24 presumably healthy volunteers. For each subject two blood samples were drawn. One using a device as described throughout this application. The second was drawn using a standard 21-gauge VeriFine safety lancet (diameter 819 μm, length 2 mm) for comparison. The testing using the device according to the invention showed reliable sample collection with 23 out of 24 successful sample collections, 19 at the first attempt and 4 at a second attempt. From one of the test subjects a blood sample of at least 1 μL was not obtained and is therefore counted as unsuccessful. The finger pricking was successful in 23 attempts, one was discarded after a first failed attempt. From the 23 successful sample collections 22 was at a first attempt and 1 at a second attempt.

The sample collection time for the device described in this application was 31 seconds with a standard deviation of 16 seconds. Considering the cases where both sampling methods were successful the pain score from the volunteers was compared. Using a blood sample device according to the present application was less painful than a finger pricking sampling. The significance of the result has been confirmed using a nonparametric Wilcoxon paired rank sum test (P=0.0003).

Of the 22 compared pairs of pain scores, 19 indicated the sampling method according to the invention as less painful. Twenty test subjects rated blood sampling using the device according to the present disclosure between and 2, which can be interpreted as, respectively, “no pain” and “mild pain”, while for finger-pricking such rating was reported by 5 of 22 volunteers. When asked for any voluntary comments, three volunteers have reported that the pain caused by finger-prick persists minutes after the procedure is completed, while the pain caused by the device according to the invention is alleviated before the procedure is completed.

By measuring the red blood cell concentration (RBC), the two types of samples were tested for bias. Such bias can result from interstitial fluid (ISF) mixing into the collected sample during collection. The RBC concentration in samples collected with the device according to the invention shows no considerable bias with respect to finger-prick samples. The CV of 13% for RBC concentrations between the compared sample types can be attributed to several factors. A study of drop-to-drop CV for hemoglobin concentration of capillary blood samples, which is directly proportional to RBC count, has shown CV of 4.4% for 20 μL drops. The sampling error should further increase with decreasing sample size. The volumetric precision of samples collected using the device according to this application, at the level of 8.2%, adds to the expected error for a single pair of compared samples. Finally, finger-prick sampling protocol inherently results in patient-to-patient variability. Namely, WHO guidelines on drawing blood state that excessive squeezing of finger should be avoided, but it can, and often is, applied to a certain degree. This can result in varying amounts of ISF being mixed with the collected samples, making the finger-prick capillary blood samples a reference with an unknown, varying component. Considering all the above, the CV of 13% in error between capillary blood samples collected using the device according to the invention and finger-prick samples is a value within the expected level of variance. It shows that the presented sampling protocol does not compromise the quality of the collected capillary blood samples.

The results of the experiments recited herein indicate that the embodiments of the present disclosure provide an efficient and minimally invasive technique for sampling blood from mammal subjects. Compared to prior art techniques, the present device may be simpler, more compact and potentially more cost-effective due to the choice of materials, the fabrication techniques involved, and the lack of complex actuators.

The inventive concept has mainly been described above with reference to a few embodiments and examples. However, as is readily appreciated by a person skilled in the art, other embodiments than the ones disclosed above are equally possible within the scope of the inventive concept as defined by the appended claims.

1. A microneedle for extracting a blood sample from skin of a mammal subject, comprising:
at least one blade being substantially flat and comprising at least one cutting edge, wherein the at least one cutting edge is configured to incise the skin of the mammal subject;
at least one microchannel comprising an opening and a passage, wherein the at least one microchannel is arranged adjacent to the at least one blade and configured to be inserted into the skin of the mammal subject together with the at least one blade, and wherein the passage is configured to transport the blood sample away from the opening via capillary action.

2. (canceled)

3. The microneedle according to claim 1, wherein the at least one blade is independently movable in relation to the at least one microchannel.

4. The microneedle according to claim 3, wherein the at least one blade is configured to be removed from the skin of the mammal subject independently from the at least one microchannel while the at least one microchannel is aspiring blood.

5. The microneedle according to claim 1, wherein the at least one microchannel further comprises an exit arranged at an end portion of the passage opposing the opening, and wherein the exit comprises a cut extending along an axial direction of the passage.

6. The microneedle according to claim 5, wherein the cut creates at least one contact point allowing the blood sample in the at least one microchannel to come into contact with a material outside of the at least one microchannel.

7. The microneedle according to claim 1, wherein a diameter (D) of the at least one microchannel is substantially smaller than a width (W) of the at least one blade.

8. The microneedle according to claim 1, wherein the at least one blade is formed by two sub blades, each comprising a cutting edge, wherein the two sub blades are joined into a tip.

9. (canceled)

10. The microneedle according to claim 8, wherein the at least one microchannel is arranged in-between the two sub blades so that each sub blade extends at least partially along a length direction of the at least one microchannel and so that the opening is aligned with the tip.

11. (canceled)

12. The microneedle according to claim 1, wherein the at least one cutting edge comprises an apex with a tip radius smaller than 20 μm, preferably smaller than 5 μm, more preferably smaller than 1 μm.

13. The microneedle according to claim 1, wherein the at least one microchannel has an inner diameter larger than 30 μm and smaller than 500 μm.

14. The microneedle according to claim 1, wherein the at least one blade has a thickness smaller than 1 mm and larger than 10 μm.

15. (canceled)

16. (canceled)

17. The microneedle according to claim 1, wherein the at least one microchannel is a closed microchannel.

18. (canceled)

19. The microneedle according to claim 1, further comprising a capillary means in connection to the at least one microchannel.

20. A device for extracting a blood sample from a mammal subject, comprising:
a microneedle according to claim 1;
a body configured to support the microneedle;
a channel arranged in the body and fluidically connected to the passage of the microneedle; and
a retaining material arranged in the body;
wherein the retaining material is fluidically connected to the channel and configured to absorb and store the blood sample transported by the passage of the microneedle to the channel.

21. The device according to claim 20, further comprising an ejecting mechanism configured to move the microneedle towards and into the skin of the mammal subject.

22. The device according to claim 21, wherein the ejecting mechanism is configured to be triggered in response to the body being pushed against the skin of the mammal subject.

23. The device according to claim 21, further comprising a retracting mechanism configured to at least move the at least one blade at least partially away from the skin after the skin has been penetrated.

24. The device according to claim 23, wherein the ejecting mechanism is configured to move the microneedle a first distance d1 and wherein the retracting mechanism is configured to at least move the at least one blade a second distance d2 opposite the first distance d1, wherein the first distance d1 is equal to or larger than the second distance d2.

25. The device according to claim 21, wherein the ejecting mechanism comprises a preloaded spring configured to move the microneedle when the body is pushed against the skin of the mammal subject.

26. The device according to claim 23, wherein the retracting mechanism comprises a spring that is loaded by the movement of the ejecting mechanism and in response moves at least part of the microneedle at least partially away from the skin of the mammal subject.

27. (canceled)

28. The device according to claim 20, wherein the device further comprises hydrophilic fibers extending from the channel into the passage.
